(19) United States
(12) Patent Application Publication
(10) Pub. No.: US 2020/0297485 A1
(43) Pub. Date: Sep. 24, 2020

(54) GUIDEWIRELESS TRANSSEPTAL DELIVERY SYSTEM FOR THERAPEUTIC DEVICES OF THE AORTIC VALVE

(71) Applicant: SYNECOR LLC, Durham, NC (US)

(72) Inventor: Richard S Stack, Chapel Hill, NC (US)

(21) Appl. No.: 16/860,015

(22) Filed: Apr. 27, 2020

Related U.S. Application Data
(60) Provisional application No. 62/811,369, filed on Feb. 27, 2019.

(51) Int. Cl. A61F 2/24; A61M 25/01

(52) U.S. Cl. CPC A61F 2/2436; A61M 25/0105; A61M 25/09; A61M 2209/04; A61M 2210/127; A61F 2/2433

(57) ABSTRACT

A method for delivering a therapeutic device to a target aortic valve site includes transseptally positioning a cable to run between a femoral artery, through the heart via an aortic valve, left ventricle, mitral valve, left atrium, and right atrium into the venous vasculature, the flexible member having a first end extending outside the body from a venous vessel superior to the heart and a second end external to the patient at the femoral artery. The aortic valve therapeutic device is attached to the cable outside the body and introduced into a femoral artery. A steerable sheath is advanced over the cable, into the venous vasculature and into the left ventricle of the heart. The aortic valve therapeutic device is pushed in a distal direction from the femoral artery while the cable is pulled from the venous vessel to advance the aortic valve therapeutic device to the target site. The sheath protects surrounding tissues from the cable during movement of the therapeutic device through the vasculature, and may be steered during final positioning to align the aortic valve therapeutic device at the target site.

This application claims the benefit of U.S. Provisional Application No. 62/811,369, filed Feb. 27, 2020, which is incorporated herein by reference.

### BACKGROUND

Commonly owned co-pending U.S. application Ser. No. 16/365,601 (Ref: AEG-1120) describes a transseptal delivery system for driving aortic valve therapeutic devices (AVTD's) into place using a combination of pulling force, pushing force, steering force and momentum. A related system that is used instead for transeptally driving mitral valve therapeutic devices into place is described in Applicant's co-pending PCT Application No. PCT/US17/62913 (Ref: ATR-820). The system and method illustrated in ATR-1120 differ from that described in ATR-820 primarily in that the aortic valve therapeutic device, once positioned in the left ventricle, is then advanced to the native aortic valve location.

Another co-pending U.S. application Ser. No. 16/578,373 (Ref: SYNC-5000) describes a transseptal delivery system and method that may be used to deliver percutaneous ventricular assist devices, or other devices such as aortic valve therapeutic devices or mitral valve therapeutic devices to their target locations.

Each of the above-described applications is incorporated herein by reference.

The present application describes a method of using a system that is similar to that described in U.S. application Ser. No. 16/578,373 for delivering an aortic valve therapeutic device to an aortic valve site using a “reverse” approach. Thus the therapeutic device is introduced into the vasculature on the arterial side (e.g. via the right femoral artery “RFA”) vs the venous side as described in each of the co-pending applications.

### BRIEF DESCRIPTION OF THE DRAWINGS

FIG. 1 is side elevation view of an exemplary low profile sheath (LPS) that may be used in the disclosed method.

FIG. 2 is a cross-section view of the LPS of FIG. 1.

### DETAILED DESCRIPTION

Reference is made to the following co-pending and commonly-owned US Applications, each of which describe components suitable for use with the presently disclosed method and each of which is incorporated herein by reference:

U.S. application Ser. No. 16/578,373 (Ref SYNC-5000R), entitled Systems and Methods for Transseptal Delivery of Percutaneous Ventricular Assist Devices and Other Non-Guidewire Based Transvascular Therapeutic Devices, which describes a transseptal delivery system and method that may be used to deliver percutaneous ventricular assist devices. Many of the components described in that application may be used in the present invention. The “cable” component, referred to as the “conveyor cable” below, may differ from the cable described in Ser. No. 16/578,373 and may instead be similar to the cable described in PCT/US17/62913 in that it has a mandrel at one end and an engageable feature such as a ball at the opposite end.

U.S. application Ser. No. 16/578,374 (Ref: SYNC-5100), entitled Conduit for Transseptal Passage of Devices to the Aorta, filed Sep. 22, 2019, describes conduits of the type that may be used for the right-to-left conduit or “RLC” described in the method of the present application.

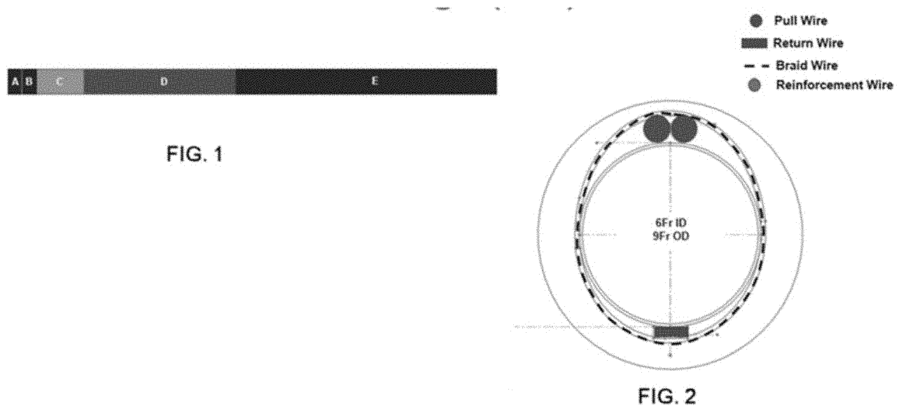

U.S. application Ser. No. 16/578,379 (Ref: SYNC-5300), entitled Instrument for Facilitating Transseptal Delivery of Cardiac Therapeutic Devices describes a protective device (referred to in that application as the “LVR”) that may be used as the low profile sheath (“LPS”) in the described method. Referring to FIGS. 1-2, the LPS may have the properties of the lower profile third embodiment described in the prior application, or it may have alternate properties. In one example shown in FIGS. 1 (in which the distal tip is denoted A) and 2, the flexural properties of the shaft may be such that the stiffness of the shaft increases by a factor of 2 from region D to region E.

With this difference, the present method also differs from the approaches described in U.S. application Ser. No. 16/365,601 and the other applications in that the LVR (which may be a lower profile LVR than the LVRs described in the prior applications, as discussed below) is introduced via a femoral vein (e.g. the RFV) as opposed to via a femoral artery.

To place an aortic valve therapeutic device at an aortic valve site using the presently described approach, the following steps may be performed:
Obtain percutaneous access of the right femoral artery (20 F sheath), the right femoral vein (16 F sheath), and the left femoral artery (8 or 9 F sheath for blood pressure and pig tail).
Make transseptal access using standard Brockenbrough technique.
Using the transseptal sheath, place a 0.035″ guide wire (Abbot Versacore) in the left atrium.
Advance a balloon catheter (12×20 mm Boston Scientific Charger) over the guide wire through the transseptal sheath and perform an atrial septostomy.
Following removal of the balloon catheter, leave the guide wire in the LA and exchange the transseptal sheath for the RLC.
Advance the RLC across the mitral valve to the left ventricular outflow tract (LVOT), watching for presence of “windshield wiper” movement of the RLC to indicate that it is unrestricted by chordae.
Advance the wire through the aortic valve to the aortic outflow tract of the descending aorta.
Temporarily exchange the RLC for the balloon catheter and advance it over the wire to the LVOT to confirm that the wire is free from chordal entrapment, pausing the advance of the balloon long enough to re-expand the septum as it passes.
The balloon catheter is exchanged for the RLC and the RLC is advanced through the heart to the descending aorta.
Insert a snare catheter through the right femoral artery and advance it first over the 0.035″ guide wire, then further up the descending aorta to capture the RLC.
The RLC is exteriorized from the right femoral artery and the wire is removed from the body through the RLC from the arterial side.
Insert the ball-shaped end of the conveyor cable through the end of the RLC exposed from the RFA and advance it until emerges from the end of the RLC protruding from the right femoral vein, leaving the cable's unsheathed 0.035″ extension exposed from the RFA.
The RLC is removed from the body, leaving the conveyor cable in place. The Conveyor Low Profile Sheath (LPS) is then inserted over the conveyor cable from the venous side and advanced to the septum.
Advance the valve delivery system over the 0.035″ conveyor cable extension (mandrel) and close the bleedback valve on the delivery system to lock it in place. Place a secondary 0.035″ locking torquer over the 0.035″ cable to eliminate the possibility of slippage in the system as it is advanced into position.
Advance the conveyor cable and attached valve delivery system into the right femoral artery via the 20 F sheath. When the valve delivery system is in the descending aorta, advance the LPS from the vena cava into position in the left ventricle.
With the LPS in place in the LV, advance the valve delivery system into position using gentle and continuous force, both pulling the conveyor cable from the venous side and advancing the valve delivery system into the arterial side until precise positioning of the prosthesis is obtained. The steering mechanism of the LPS is used to precisely align the prosthesis for deployment.
Once the operator is satisfied with the deployment of the prosthetic valve, the conveyor cable and valve delivery system are removed from the arterial side and the LPS is removed from the venous side.

Each of the patents and applications referred to herein, including for purposes of priority, are incorporated herein by reference.

What is claimed is:

1. A method for delivering a therapeutic device to a target aortic valve site, comprising:
(a) positioning a cable in a body of a patient to run between a femoral artery, through the heart via an aortic valve, left ventricle, mitral valve, left atrium, and right atrium into the venous vasculature, the flexible member having a first end extending outside the body from a venous vessel superior to the heart and a second end external to the patient at the femoral artery;
(b) releasably connecting an aortic valve therapeutic device to the cable and introduce the aortic valve therapeutic device into a femoral artery;
(c) advancing a sheath over the cable into the venous vasculature and into the left ventricle of the heart;
(d) pushing the aortic valve therapeutic device in a distal direction from the femoral artery while pulling the cable from the venous vessel to advance the aortic valve therapeutic device to the target site.

2. The method of claim 1, further including steering the LPS during step (d) to align the aortic valve therapeutic device at the target site.
